(19) United States
(12) Patent Application Publication
(10) Pub. No.: US 2012/0316457 A1
(43) Pub. Date: Dec. 13, 2012

(54) Acupuncture Meridian Measurement System

(75) Inventors: Hao Meng, Beijing (CN); Charles Lee, Taipei (TW)

(73) Assignee: ANALOG DEVICES, INC., Norwood, MA (US)

(21) Appl. No.: 13/279,742

(22) Filed: Oct. 24, 2011

(30) Foreign Application Priority Data
Jun. 8, 2011 (CN) 201120189272.2

(51) Int. Cl. A61B 5/053

(52) U.S. Cl. 600/548

(57) ABSTRACT

The present disclosure relates to an acupuncture meridian measurement system. The acupuncture meridian measurement system comprises: a signal generator configured to generate an alternating current excitation signal; an impedance measurer coupled to the signal generator and configured to measure a biological impedance between a fixed point to be measured and at least one selected point of an object; an acupuncture point determiner coupled to the impedance measurer and configured to locate an acupuncture point based on the measured biological impedance, wherein, the determined acupuncture point is a selected point, the biological impedance between which and the fixed point is less than a threshold. A technical problem solved by an embodiment of the present disclosure is to realize an accurate location of the acupuncture meridian. A use of an embodiment of the present disclosure is to locate acupuncture meridian and Yuan acupuncture points.

### PRIORITY

The present application claims priority from Chinese Patent Application No. 201120189272.2, filed on Jun. 8, 2011, and naming Hao Meng and Charles Lee as inventors, the disclosure of which is incorporated herein, in its entirety, by reference.

### TECHNICAL FIELD

The present utility model relates to an acupuncture meridian measurement system.

### BACKGROUND ART

According to Chinese traditional medical theories, meridians are channels for circulations of “qi” and blood throughout a biological body. Take a human body as an example, except for the conception vessel (CV) (“ren meridian”) and the governor vessel (GV) (“du meridian”), there are 12 major symmetrically distributed meridians, such as lung meridian, stomach meridian, heart meridian, and so on.

Each meridian has a corresponding acupuncture point. Various attempts have been made in order to objectively locate meridians and their corresponding acupuncture points. It has been found that the impedance of an acupuncture meridian is significantly lower than that of a part other than acupuncture meridian. In addition, the impedance of an acupuncture point obviously decreases as compared to other parts of a biological body. Thus, a device for locating and monitoring meridians using electrical means and a method thereof are developed.

Usually, skin and acupuncture meridian of a biological body can be modeled as a complex impedance. As shown in FIG. 1, a biological body to be measured is modeled into a RC model, wherein, the impedance R1 and the capacitive reactance C1 can correspond to stratum corneum and epidermis respectively, while R2 can correspond to dermis.

In existing impedance measuring method, a direct current (DC) excitation is applied to measure a biological impedance of a position to be measured. In the technology for measuring an impedance of a biological body as described above, a problem occurs that the capacitive reactance in the model cannot be effectively discharged, since a DC excitation is applied. As such, the accuracy of the measurement of the biological impedance is affected.

Furthermore, since a delay required for waiting the capacitive reactance to be discharged is present, the speed of the measurement is also affected. Thus, it is further adversely affect the location and monitoring of acupuncture meridian.

Therefore, in the art, it is desirable to provide a technology for more accurately locating an acupuncture meridian.

### SUMMARY OF THE INVENTION

One objective of the present utility model is to provide an improved acupuncture meridian measurement system that can achieve an accurate location of an acupuncture meridian.

Another purpose of the present utility model is to provide an improved acupuncture meridian measurement system that can enhance the speed of the meridians measurement.

An acupuncture meridian has acupuncture points for controlling “qi”. The “Yuan” (source) acupuncture points are acupuncture points on the acupuncture meridian where the “Yuan Qi” of a body reaches the surface. The “Yuan” acupuncture points can typically characterize the statuses of corresponding acupuncture meridian. Generally, as for the “Yin” meridians, the “Yuan” acupuncture point is the third acupuncture point from the extremity on the channel thereof; as for the “Yang” meridians, the “Yuan” acupuncture point is the fourth acupuncture point from the extremity on the channel thereof.

The present utility model can measure an impedance of a biological body so as to locate an acupuncture meridian and an acupuncture point, such as the “Yuan” acupuncture point.

According to an embodiment of the present disclosure, an acupuncture meridian measurement system is provided, which comprises: a signal generator configured to generate an alternating current (AC) excitation signal; an impedance measurer configured to measure a biological impedance between a fixed point and at least one selected point of an object to be measured using the AC excitation signal, wherein the impedance measurer is coupled to the signal generator; and an acupuncture point determiner configured to determine the selected point, the biological impedance between which and the fixed point is less than a predetermined threshold, based on the determined biological impedances, so as to locate an acupuncture point. The acupuncture point determiner is coupled to the impedance measurer.

According to an embodiment of the present disclosure, the signal generator is configured to generate a sweep frequency signal as the AC excitation signal.

According to an embodiment of the present disclosure, the signal generator is configured to generate a sweep frequency signal having a frequency less than 100 Hz.

According to an embodiment of the present disclosure, the signal generator is configured to generate a sweep frequency signal having a frequency range from 50 through 95 Hz.

According to an embodiment of the present disclosure, the impedance measurer further comprises: an operation amplifier that is coupled to the biological impedance of an object to be measured; and a feedback resistor whose resistance is determined according a biological impedance of an acupuncture point to be measured and which is coupled between an input and an output of the operation amplifier.

According to an embodiment of the present disclosure, the acupuncture point determiner is configured to determine the acupuncture point corresponding to the selected point, the biological impedance between which and said fixed point is less than 200 kΩ, as a “Yuan” acupuncture point.

According to an embodiment of the present disclosure, the acupuncture point determiner is configured to determine the acupuncture point corresponding to the selected point, the biological impedance between which and said fixed point is less than a predetermined threshold within a range of 100-200 kΩ, as a “Yuan” acupuncture point.

According to an embodiment of the present disclosure, the acupuncture point determiner is configured to determine the acupuncture point corresponding to the selected point, the biological impedance between which and said fixed point is less than a predetermined threshold within a range of 200-450 kΩ, as a “Yuan” acupuncture point.

According to an embodiment of the present disclosure, the acupuncture meridian measurement system is configured to be calibrated with a calibration impedance of 100 kΩ.

According to an embodiment of the present disclosure, the impedance measurer is configured to measure the biological impedance of the object to be measured in accordance to an average value calculated for a plurality of discrete sweep frequency points of the sweep frequency signal.

According to an embodiment of the present disclosure, the acupuncture meridian measurement system further comprises a signal processor configured to process a signal outputted from the impedance measurer, wherein said signal processor is coupled between the impedance measurer and the acupuncture point determiner.

According to an embodiment of the present disclosure, the signal processor further comprises: an analog-to-digital converter that is configured to convert an analog signal representing the measured biological impedance into a digital signal and is coupled to an output of the impedance measurer; and, a Fourier transformer that is configured to perform Fourier transformation on the digital signal and is coupled between an output of the analog-to-digital converter and an input of the acupuncture point determiner.

According to an embodiment of the present disclosure, the signal processor further comprises a programmable gain amplifier that is coupled between an output of the impedance measurer and an input of the analogy-to-digital converter.

According to an embodiment of the present disclosure, the signal processor further comprises a low-frequency filter that is coupled between an output of the programmable gain amplifier and an input of the analog-to-digital converter.

According to an embodiment of the present disclosure, the acupuncture meridian measurement system further comprises a micro-controlling unit configured to calculate the amplitude of an impedance of an object to be measured based on the output of the Fourier transformer, wherein, said micro-controlling unit is coupled between an output of the Fourier transformer and an input of the acupuncture point determiner.

According to an embodiment of the present disclosure, the acupuncture meridian measurement system further comprises a computer coupled to an output of the Fourier transformer, wherein, said computer comprises a processor configured to calculate the amplitude of an impedance of an object to be measured based on the output of the Fourier transformer.

According to an embodiment of the disclosure, the acupuncture meridian measurement system comprises a computer.

According to an embodiment of the present disclosure, the acupuncture point determiner is provided in the computer.

According to an embodiment of the present disclosure, the computer comprises a graphic user interface (GUI) that presents a measurement result of the acupuncture meridian measurement system.

One technical effect obtained by the acupuncture meridian measurement system according to an embodiment of the present utility model is that a capacitive reactance of an object to be measured can be effectively discharged by applying a voltage excitation using an AC signal and thus the accuracy of the biological impedance measurement can be enhanced, thereby the acupuncture meridian can be accurately located and monitored, particularly those acupuncture points corresponding to the acupuncture meridian, such as the “Yuan” acupuncture points.

Another technical effect obtained by the acupuncture meridian measurement system according to an embodiment of the present utility model is that the accuracy of the biological impedance measurement can be further improved using an average value of the impedance values measured at a plurality of sweep frequency points by applying a voltage excitation using an AC sweep frequency signal, thereby the acupuncture meridian can be more accurately located and monitored, particularly those acupuncture points corresponding to the acupuncture meridian, such as the “Yuan” acupuncture points.

A further technical effect obtained by the acupuncture meridian measurement system according to an embodiment of the present utility model is that a delay required for waiting the capacitive reactance of an object to be measured to be discharged is eliminated with a voltage excitation applied using an AC signal, thereby the speed of the acupuncture meridian measurement can be enhanced.

### BRIEF DESCRIPTION OF THE DRAWINGS

For a better understanding of the features of the present utility model, the accompanying drawings are described below. It is noted that, however, the drawings are merely illustrating the detailed embodiments of the present utility model but should not be construed as limiting the present utility model, since the present utility model can incorporate other equivalent embodiments.

FIG. 1 illustrates an RC model for biological impedance.

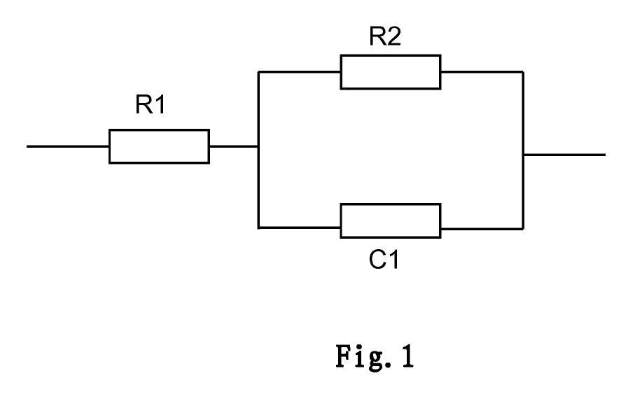

FIG. 2 illustrates a block diagram of the acupuncture meridian measurement system according to an embodiment of the present utility model.

FIG. 3 illustrates a block diagram of the acupuncture meridian measurement system according to another embodiment of the present utility model.

FIG. 4 illustrates a block diagram of the acupuncture meridian measurement system according to a further embodiment of the present utility model.

FIG. 5 illustrates a schematic perspective view of the portable acupuncture meridian measurement system according to an embodiment of the present utility model.

### DETAILED DESCRIPTION OF SPECIFIC EMBODIMENTS

The embodiments of the present utility model can provide an acupuncture meridian measurement system having improved measurement accuracy. The embodiments of the present utility model can also provide an acupuncture meridian measurement system having enhanced measurement speed. The embodiments can generate an AC sweep frequency signal using a signal generator, and apply a voltage excitation using the AC sweep frequency signal, and measure the biological impedance of an object to be measured using an impedance measurer. Since a capacitive reactance of an object to be measured can be effectively discharged by an AC sweep frequency signal, the accuracy of the biological impedance measurement can be improved. The embodiments can perform processes such as gain, filter, analog-to-digital conversion (ADC), Fourier transformation (FT) on the measured signal so as to further enhance the measurement accuracy. A delay required for waiting the capacitive reactance of an object to be measured to be discharged is eliminated with a voltage excitation applied using an AC signal, thereby the speed of the acupuncture meridian measurement can be enhanced.

The present utility model will be described with reference to the accompanying drawings.

### First Embodiment

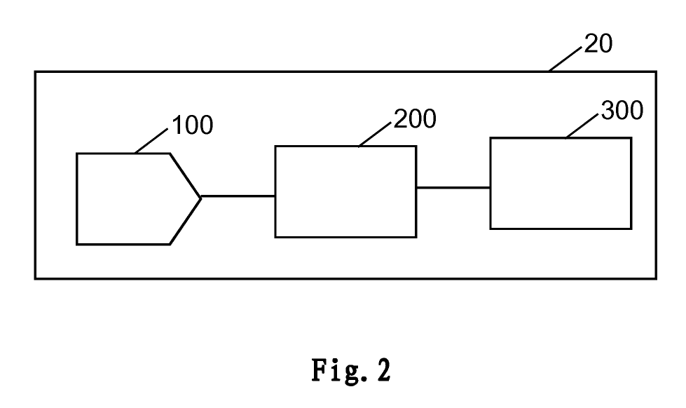

FIG. 2 illustrates a block diagram of the acupuncture meridian measurement system according to the first embodiment of the present utility model.

As illustrated in FIG. 2, the acupuncture meridian measurement system 20 according to the present embodiment can comprise a signal generator 100, an impedance measurer 200 and an acupuncture point determiner 300.

The signal generator 100 generates and provides an AC excitation signal. Optionally, the signal generator 100 can generate and provide an AC sweep frequency excitation signal (herein also referred to as low frequency scanning excitation signal).

For example, the signal generator 100 can be a direct digital frequency synthesizer (DDS). The direct digital frequency synthesizer can employ a system clock or an internal clock produced by an internal oscillator (not shown). The frequency of the sweep frequency excitation signal provided by the signal generator 100 can be adjusted based on a rough range of the impedance value of an object to be measured. In this embodiment, the frequency of the sweep frequency excitation signal is less than 100 Hz. Preferably, the frequency of the sweep frequency excitation signal is in a range of 50-95 Hz.

The impedance measurer 200 is coupled to the signal generator 100 and measures the biological impedance of the object to be measured under the sweep frequency excitation signal provided by the signal generator 100. A value of the measured biological impedance can be a resistance numerical value measured using current-voltage characteristics or a code from which a corresponding impedance value can be derived through calculation, such as a complex impedance output code composed of a real part and an imaginary part that is specifically described below in the third embodiment.

The acupuncture point determiner 300 is coupled to the impedance measurer 200 and locates an acupuncture point based on the measured biological impedance. The biological impedance of an acupuncture point on acupuncture meridian is less than a predetermined threshold. Typically, the biological impedance of the “Yuan” acupuncture points under a normal condition is less than 200 kΩ.

Those skilled in the art should appreciate that the predetermined threshold can be adjusted accordingly when the environmental conditions affecting the measurement change. For example, in the case of a relatively low humidity, the measured biological impedance may rise. In such case, the above predetermined threshold can be appropriately adjusted upward to be a predetermined threshold within 200-450 kΩ, such as 250 kΩ, 300 kΩ, 350 kΩ, 400 kΩ or 450 kΩ.

On the other hand, in the case of a relatively high humidity, the measured biological impedance may drop. In such case, the above predetermined threshold can be appropriately adjusted downward to be a predetermined threshold within 100-200 kΩ, such as 150 kΩ or 100 kΩ.

However, under a bad condition, the biological impedance of the “Yuan” acupuncture point will be significantly lower than the aforementioned predetermined threshold, even lower than 100 kΩ, such as be 75 kΩ or even less.

In this embodiment, it is assumed that the measurement is carried out under a typical environmental condition and a position whose biological impedance is less than 200 kΩ is determined as the “Yuan” acupuncture point.

Generally, the acupuncture meridian measurement system 20 needs to be calibrated with regard to a known range of the impedance of an object to be measured and then can be used to perform an effective measurement. In this embodiment, the acupuncture meridian measurement system 20 is calibrated using calibration impedance (not shown). The value of the calibration impedance can be an impedance value selected from a range of 100-450 kΩ. For example, the value of the calibration impedance may be 100 kΩ.

As an example, the acupuncture meridian measurement system 20 may have an operation voltage of AC 2V and an operation current less than 10 μA.

In practical operation, a measurement terminal (not shown) of the acupuncture meridian measurement system 20 can be attached to the object to be measured. For example, one terminal thereof can be held by the object to be measured, while the other terminal can be moved along the object to be measured to measure the impedance between a fixed point and a selected point, thereby locating the acupuncture point, such as the “Yuan” acupuncture point. As an example, one terminal (not shown) of the acupuncture meridian measurement system 20 can be held and retained fixed by a hand of a human as the object to be measured, while the other terminal (not shown) of the acupuncture meridian measurement system 20 can be moved along the surface of the human body, so as to measure the impedance between the point (a fixed point) where the one terminal contacts the hand and the point (a selected point) where the other terminal contacts the surface of the human body. The determined acupuncture point is a selected point of which the biological impedance to the fixed point is less than the predetermined threshold.

The acupuncture meridian measurement system 20 can include a computer (not shown) such as a desk computer, a portable computer, a server and so on. In this case, the acupuncture point determiner 300 that is provided within a computer can be implemented. That is, the acupuncture points such as the “Yuan” acupuncture point can be located with the computer based on the biological impedance measured by the acupuncture meridian measurement system 20. Further, the computer can comprise a graphic user interface that presents a measurement result of the acupuncture meridian measurement system 20, so as to improve system interactivity.

In this embodiment, a voltage excitation is applied using an AC sweep frequency signal and the biological impedance of the object to be measured is measured by the impedance determiner. Since the capacitive reactance of the object to be measured can be effectively discharged with the AC sweep frequency signal, the accuracy of the biological impedance measurement is improved. A delay required for waiting the capacitive reactance of the object to be measured to be discharged is eliminated with a voltage excitation applied using the AC signal, thereby the speed of the acupuncture meridian measurement can be enhanced.

In this embodiment, the impedance can be measured with regard to a plurality of discrete sweep frequency points of the sweep frequency excitation signal and an average value of the measured impedances can be obtained, thereby the accuracy of the measurement can be further improved.

### Second Embodiment

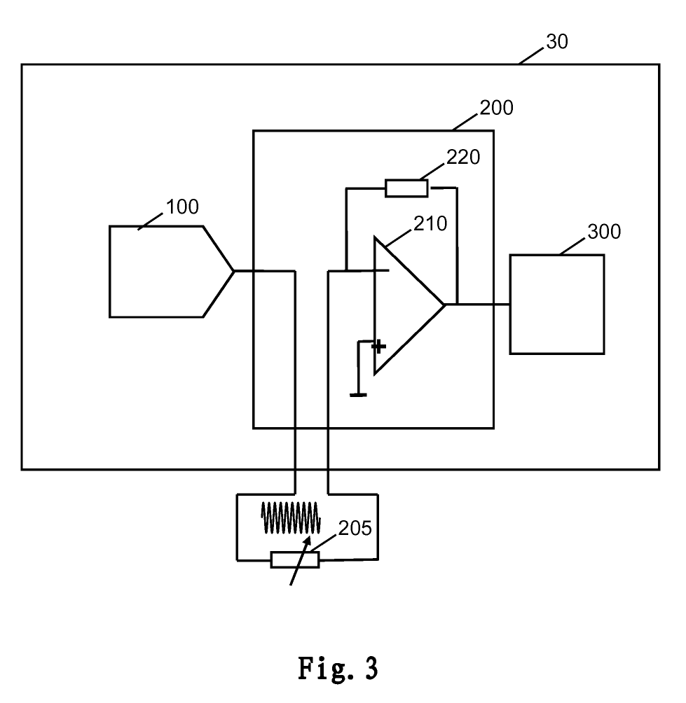

FIG. 3 illustrates a block diagram of the acupuncture meridian measurement system according to the second embodiment of the present utility mode. In this embodiment, similar reference numbers are used for denoting similar components as in the first embodiment and the description thereof may be omitted herein.

As shown in FIG. 3, the acupuncture meridian measurement system 30 according to the second embodiment can include a signal generator 100, an impedance measurer 200 and an acupuncture point determiner 300.

The configuration and operation of the signal generator 100 and the acupuncture point determiner 300 of the second embodiment are similar as that of the first embodiment, and the description thereof is omitted for the sake of concision.

The impedance measurer 200 can include an operation amplifier 210 and a feedback resistor 220. As shown, the operation amplifier 210 is coupled to biological impedance 205 of an object to be measured. A voltage excitation is applied to the biological impedance 205 of the object to be measured with a low frequency scanning excitation signal outputted by the signal generator 100, as shown. Response signal current generated on the biological impedance 205 of the object to be measured flows into a negative input of the operation amplifier 210 and a voltage signal is generated at an output of the operation amplifier 210. Thus, the value of the biological impedance 205 of the object to be measured can be measured using the current-voltage characteristics.

In this embodiment, as an example, the feedback resistor 220 is coupled between a negative input and an output of the operation amplifier 210. A positive input of the operation amplifier 210 can be coupled to a virtual ground (for example, VDD/2).

Similar to the first embodiment, in this embodiment, a position whose biological impedance is less than 200 kΩ is determined as the “Yuan” acupuncture point. Likewise, a predetermined threshold used for determining the “Yuan” acupuncture point can be appropriately adjusted in a similar way as described in first embodiment. Further, the acupuncture meridian measurement system 30 is calibrated using a calibration impedance (not shown). The value of the calibration impedance can be a value selected from a range from 100 kΩ to 450 kΩ. For example, the value of the calibration impedance may be 100 kΩ.

The resistance of the feedback resistor 220 is determined based on the value of the biological impedance of the acupuncture point to be measured. In this embodiment, in the case of using a current limited resistor of 400 kΩ, the impedance value of the feedback resistor 220 can be set to 500 kΩ.

As an example, the acupuncture meridian measurement system 30 may have an operation voltage (VDD) of AC 2V and an operation current less than 10 μA.

In practical operation, a measurement terminal (not shown) of the acupuncture meridian measurement system 30 can be attached to the object to be measured. For example, one terminal thereof can be held by the object to be measured, while the other terminal can be moved along the object to be measured to measure the impedance between a fixed point and a selected point, thereby locating the acupuncture point, such as the “Yuan” acupuncture point. As an example, one terminal (not shown) of the acupuncture meridian measurement system 30 can be held and retained fixed by a hand of a human who servers as the object to be measured, while the other terminal (not shown) of the acupuncture meridian measurement system 30 can be moved along the surface of the human body, so as to measure the impedance between the point (a fixed point) where the one terminal contacts the hand and the point (a selected point) where the other terminal contacts the surface of the human body. The determined acupuncture point is a selected point of which the biological impedance to the fixed point is less than the predetermined threshold.

The acupuncture meridian measurement system 30 can include a computer (not shown) such as a desk computer, a portable computer, a server and so on. In this case, the acupuncture point determiner 300 that is provided within a computer can be implemented. That is, the acupuncture points such as the “Yuan” acupuncture point can be located based on the biological impedance measured by the acupuncture measurement system 30 on a computer. Further, the computer can comprise a graphic user interface that presents a measurement result of the acupuncture meridian measurement system 30, so as to improve system interactivity.

In this embodiment, a voltage excitation is applied using an AC sweep frequency signal and the biological impedance of the object to be measured is measured by the impedance determiner. Since the capacitive reactance of the object to be measured can be effectively discharged with the AC sweep frequency signal, the accuracy of the biological impedance measurement is improved. A delay required for waiting the capacitive reactance of the object to be measured to be discharged is eliminated with a voltage excitation applied using the AC signal, thereby the speed of the acupuncture meridian measurement can be enhanced.

In this embodiment, the impedance can be measured with regard to a plurality of discrete sweep frequency points of the sweep frequency excitation signal and an average value of the measured impedances can be obtained, thereby the accuracy of the measurement can be further improved.

### Third Embodiment

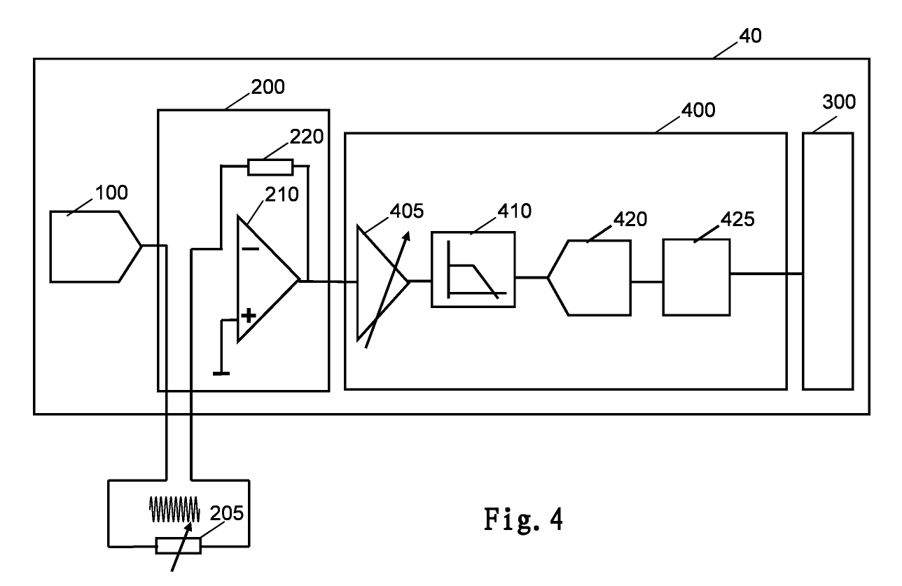

FIG. 4 illustrates a block diagram of the acupuncture meridian measurement system according to the third embodiment of the present utility model. In this embodiment, similar reference numbers are used for denoting similar components as in the second embodiment and the description thereof is omitted.

As shown in FIG. 4, the acupuncture meridian measurement system 40 according to the third embodiment can include a signal generator 100, an impedance measurer 200, an acupuncture point determiner 300 and a signal processor 400.

The configuration and operation of the signal generator 100, the impedance measurer 200 and the acupuncture point determiner 300 of the third embodiment are similar as that of the second embodiment, and the description thereof is omitted for the sake of concision.

Different from the second embodiment, the acupuncture meridian measurement system 40 of the third embodiment further comprises the signal processor 400 that is coupled between the impedance measurer 200 and the acupuncture point determiner 300. Signals outputted from the impedance determiner 200 can be processed by the signal processor 400.

As shown, the signal processor 400 can include an analog-to-digital converter 420 that is coupled to an output of the impedance measurer 200. The impedance measurer 200 can output an analog signal representing the value of the measured biological impedance, such as the voltage signal described in the second embodiment. The analog-to-digital converter 420 is configured to convert the analog signal to a digital signal. That is, the analog-to-digital converter 420 samples the response signal of the complex impedance of the object to be measured for the sweep frequency signal excitation generated by the frequency generator 100.

The signal processor 400 can further include a Fourier transformer 425 that is coupled to an output of the analog-to-digital converter 420. The Fourier transformer 425 is configured to perform Fourier transformation (FT) on the digital signal converted by the analog-to-digital converter 420. For example, a real (R) data word and an imaginary (I) data word is returned at each frequency using a discrete Fourier transformation (DFT) algorithm. Note that, the present utility model can also employ other algorithms, such as fast Fourier transformation algorithm (FFT). Based on the real data word and the imaginary data word at each frequency point as described above, the FT amplitude of this point can be calculated. This calculation can be performed by a micro-controlling unit (MCU) (not shown) that is coupled to the Fourier transformer 425 and is included in the acupuncture meridian measurement system 40, or can be performed by a processor of the computer (not shown) that is coupled to the acupuncture meridian measurement system 40.

For example, the FT amplitude can be calculated using the following formula:

Amplitude=\sqrt{R²+I²} (1)

In order to convert this amplitude into impedance, it must be multiplied by a coefficient of proportionality that is called a gain factor. The gain factor is calculated using the calibration resistance during the system calibration. For example, the gain factor can be calculated using the following formula:

$$GainFactor = \frac{( \frac{1}{Impedance} )}{Amplitude} \qquad ( 2 )$$

After the gain factor is calculated, it can be used to calculate the impedance of any object to be measured that is accessible to the acupuncture meridian measurement system 40.

For example, the impedance at each frequency point can be calculated as follows:

$$Impedance = \frac{1}{GainFactor \times Amplitude} \qquad ( 3 )$$

Similar to the first embodiment, in this embodiment, a position whose biological impedance is less than 200 kΩ is determined as the “Yuan” acupuncture point. Likewise, a predetermined threshold used for determining the “Yuan” acupuncture point can be appropriately adjusted in a similar way as described in the first embodiment. Further, the acupuncture meridian measurement system 40 can be calibrated using calibration impedance (not shown). The value of the calibration impedance can be a value selected from a range from 100 kΩ to 450 kΩ. For example, the value of the calibration impedance may be 100 kΩ. In this embodiment, the impedance calculated as above is used for calibration.

In order to further improve the accuracy of the measurement, optionally, the signal processor 400 can further include a programmable gain amplifier 405 that is coupled to an output of the operation amplifier 210. In this embodiment, in the case where the gain of the gain amplifier 405 is 1, the resistance of the feedback resistor 220 can correspond to the resistance of the calibration impedance.

Optionally, the signal processor 400 can further comprise a low frequency filter 410 that is coupled between the programmable gain amplifier 405 and the analog-to-digital converter 420. The low frequency filter 410 can be used to remove noises such as aliasing noises from the signal.

As an example, the acupuncture meridian measurement system 40 may have an operation voltage of AC 2V and an operation current less than 10 μA.

In practical operation, a measurement terminal (not shown) of the acupuncture meridian measurement system 40 can be attached to the object to be measured. For example, one terminal thereof can be held by the object to be measured, while the other terminal can be moved along the object to be measured to measure the impedance between a fixed point and a selected point, thereby locating the acupuncture point, such as the “Yuan” acupuncture point. As an example, one terminal (not shown) of the acupuncture meridian measurement system 40 can be held and retained fixed by a hand of a human who servers as the object to be measured, while the other terminal (not shown) of the acupuncture meridian measurement system 40 can be moved along the surface of the human body, so as to measure the impedance between the point (a fixed point) where the one terminal contacts the hand and the point (a selected point) where the other terminal contacts the surface of the human body. The determined acupuncture point is a selected point of which the biological impedance to the fixed point is less than the predetermined threshold.

The acupuncture meridian measurement system 40 can include a computer (not shown) such as a desk computer, a portable computer, a server and so on. In this case, the acupuncture point determiner 300 that is provided within a computer can be implemented. That is, the acupuncture points such as the “Yuan” acupuncture point can be located based on the biological impedance measured by the acupuncture meridian measurement system 40 on a computer. Further, the computer can comprise a graphic user interface that presents a measurement result of the acupuncture meridian measurement system 40, so as to improve system interactivity.

In this embodiment, a voltage excitation is applied using an AC sweep frequency signal and the biological impedance of the object to be measured is measured by the impedance determiner. Since the capacitive reactance of the object to be measured can be effectively discharged with the AC sweep frequency signal, the accuracy of the biological impedance measurement is improved. A delay required for waiting the capacitive reactance of the object to be measured to be discharged is eliminated with a voltage excitation applied using the AC signal, thereby the speed of the acupuncture meridian measurement can be enhanced.

In this embodiment, the impedance can be measured with regard to a plurality of discrete sweep frequency points of the sweep frequency excitation signal and an average value of the measured impedances can be obtained, thereby the accuracy of the measurement can be further improved.

Furthermore, in this embodiment, processes such as gain, filter, analog-to-digital conversion, FT, and so on can be performed on the measured signal, so as to further improve the accuracy of the measurement.

The acupuncture meridian measurement system according to this disclosure can be applied to locate and monitor the acupuncture meridian and the “Yuan” acupuncture points of a human body. In addition, the acupuncture meridian measurement system of this disclosure can also be applicable to other animals and plants so as to monitor their body status or freshness respectively.

### Example

The acupuncture meridian measurement system according to the above embodiments of the present utility model can be implemented in various forms.

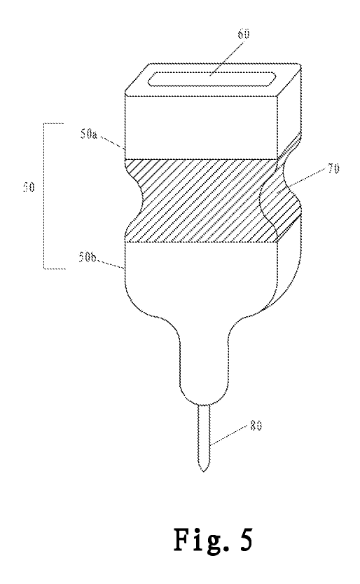

As an example, FIG. 5 illustrates a schematic perspective view of the portable acupuncture meridian measurement system according to an embodiment of the present utility model.

As shown, the portable acupuncture meridian measurement system according to the embodiment of the present utility model can comprise a housing 50. For example, the housing 50 is made of plastic or similar materials. As shown, the housing 50 can comprise two separated portions 50a and 50b.

Between the two portions 50a and 50b of the housing 50, there is provided a terminal 70 (i.e. handheld terminal) of the acupuncture meridian measurement system. As described above, at the time of measurement, the handheld terminal is held by a hand and retained immobile. The terminal 70 can be designed to be of a shape that is convenient for being held, such as the curved shape shown in the figure. In this embodiment, the terminal 70 can be made of metal. The handheld terminal can correspond to the aforementioned fixed point. Note that, the hatched part in the figure is merely for explanation, but not intended to limit the terminal 70.

In practice, the housing 50 and the terminal 70 together form an encapsulation of the portable acupuncture meridian measurement system according to the embodiment of the present utility model. All or a part of the components of the portable acupuncture meridian measurement system described in the first to third embodiments can be accommodated in the encapsulation.

Terminal 80 (i.e. measurement probe of the acupuncture meridian measurement system) extending outwardly from the housing 50 can be provided at the end of the portion 50B of the housing 50. As described above, at the time of measurement, the measurement probe can be moved along the surface of a human body. The measurement probe can correspond to the aforementioned selected point.

Optionally, in the housing 50, for example, on the top surface of the portion 50a of the housing 50, a display portion, such as a LCD display screen, can be provided for displaying measurement results or the like. The present utility model, however, is not limited to this; the display portion can be arranged at any position convenient for viewing by a user, such as in the front surface of the portion 50a.

With the portable structure shown in FIG. 5, the miniature of the acupuncture meridian measurement system can be realized, such that it is easy for carrying and capable of measuring acupuncture meridians at any time.

In addition, since the handheld terminal is integrated into the encapsulation, it can be held by one hand and move the acupuncture meridian measurement system, thereby conveniently realizing a self-service acupuncture meridian measurement. Thus, the usability of the portable acupuncture meridian measurement system can be significantly improved.

Various features and aspects of the present utility model have been described and illustrated with regard to specific embodiments in an exemplary manner but not in a limiting manner. Those skilled in the art will appreciate that a part of or all components of the aforementioned acupuncture meridian measurement system can be implemented in hardware, software or firmware. Furthermore, those skilled in the art may also recognize that alternative implementation and various modifications to the disclosed embodiments are within the range and expectation of the present disclosure. Therefore, the present utility model is intended to be merely limited by the attached claims.

What is claimed is:

1. An acupuncture meridian measurement system comprising:
a signal generator configured to generates an alternating current excitation signal;
an impedance measurer coupled to said signal generator, and configured to measure a biological impedance between a fixed point and at least one selected point of an object to be measured using said alternating current excitation signal; and
an acupuncture point determiner coupled to said impedance measurer, and configured to determine the selected point, the biological impedance between which and the fixed point is less than a predetermined threshold, based on the measured biological impedance, so as to locate an acupuncture point.

2. The acupuncture meridian measurement system according claim 1, wherein said signal generator is configured to generate a sweep frequency signal as said alternating current excitation signal.

3. The acupuncture meridian measurement system according claim 2, wherein said signal generator is configured to generate said sweep frequency signal of a frequency less than 100 Hz.

4. The acupuncture meridian measurement system according claim 2, wherein said signal generator is configured to generate said sweep frequency signal of a frequency range from 50 Hz to 95 Hz.

5. The acupuncture meridian measurement system according to claim 1, wherein said impedance measurer further comprises:
an operation amplifier that is coupled to biological impedance of the object to be measured; and
a feedback resistor whose resistance is determined according to a biological impedance of an acupuncture point to be measured, said feedback resistor being coupled between an output and an input of said operation amplifier.

6. The acupuncture meridian measurement system according to claim 1, wherein said acupuncture point determiner is configured to determine the acupuncture point corresponding to the selected point, the biological impedance between which and said fixed point is less than 200 kΩ, as a “Yuan” acupuncture point.

7. The acupuncture meridian measurement system according to claim 1, wherein said acupuncture point determiner is configured to determine the acupuncture point corresponding to the selected point, the biological impedance between which and said fixed point is less than a predetermined threshold within a range of 100-200 kΩ, as a “Yuan” acupuncture point.

8. The acupuncture meridian measurement system according to claim 1, wherein said acupuncture point determiner is configured to determine the acupuncture point corresponding to the selected point, the biological impedance between which and said fixed point is less than a predetermined threshold within a range of 200-450 kΩ, as a “Yuan” acupuncture point.

9. The acupuncture meridian measurement system according to claim 1, wherein said acupuncture meridian measurement system is configured to be calibrated with a calibration impedance of 100 kΩ.

10. The acupuncture meridian measurement system according to claim 2, wherein said impedance measurer is configured to measure the biological impedance of the object to be measured according to an average value calculated for a plurality of discrete sweep frequency points of said sweep frequency signal.

11. The acupuncture meridian measurement system according to claim 1, further comprising:
a signal processor coupled between said impedance measurer and said acupuncture point determiner and configured to process a signal outputted from said impedance measurer.

12. The acupuncture meridian measurement system according to claim 11, wherein said signal processor further comprises:
an analog-to-digital converter coupled to an output of said impedance measurer and configured to convert an analog signal representing the measured biological impedance into a digital signal; and
a Fourier transformer coupled between an output of said analog-to-digital converter and an input of said acupuncture point determiner and configured to perform Fourier transformation on said digital signal.

13. The acupuncture meridian measurement system according to claim 12, wherein said signal processor further comprises:
a programmable gain amplifier coupled between an output of said impedance measurer and an input of said analog-to-digital converter.

14. The acupuncture meridian measurement system according to claim 13, wherein said signal processor further comprises:
a low-frequency filter coupled between an output of said programmable gain amplifier and an input of said analog-to-digital converter.

15. The acupuncture meridian measurement system according to claim 12, further comprising:
a micro-controlling unit configured to calculate the amplitude of an impedance of the object to be measured based on an output of said Fourier transformer, said micro-controlling unit being coupled between an output of said Fourier transformer and an input of said acupuncture point determiner.

16. The acupuncture meridian measurement system according to claim 12, further comprising a computer coupled to an output of said Fourier transformer, wherein said computer comprises a processor configured to calculate the amplitude of the impedance of the object to be measured based on an output of said Fourier transformer.

17. The acupuncture meridian measurement system according to claim 1, wherein said acupuncture meridian measurement system further comprises a computer.

18. The acupuncture meridian measurement system according to claim 17, wherein said acupuncture point determiner is provided within said computer.

19. The acupuncture meridian measurement system according to claim 17, wherein said computer further comprises a graphic user interface for presenting a measurement result of said acupuncture meridian measurement system.

20. The acupuncture meridian measurement system according to claim 1, wherein said acupuncture meridian measurement system is a portable device and further comprises:
an encapsulation for accommodating said signal generator, said impedance measurer and said Yuan acupuncture point determiner, wherein said encapsulation comprises:
a housing including a first portion and a second portion;
a terminal of said acupuncture meridian measurement system corresponding to said fixed point and having a configuration convenient to be held, said terminal being provided between said first portion and said second portion,
wherein, the other terminal of said acupuncture meridian measurement system corresponding to said selected point has a configuration extending from an end portion of said second portion of said housing.

21. The acupuncture meridian measurement system according to claim 20, wherein said acupuncture meridian measurement system further comprises:
a display portion provided in said first portion of said housing.
